(19) United States
(12) Patent Application Publication
(10) Pub. No.: US 2023/0302207 A1
(43) Pub. Date: Sep. 28, 2023

(54) DIALYSIS SYSTEM HAVING THERMOELECTRIC HEATING

(71) Applicants: BAXTER INTERNATIONAL INC., Deerfietd, IL (US); BAXTER HEALTHCARE SA, Glattpark (Opfikon) (CH)

(72) Inventors: Michael Pettersson, Malmö (SE); Jimmie Marcus Axel Hansson, Limhamn (SE); Oskar Erik Frode Styrbjörn Fällman, Lund (SE)

(21) Appl. No.: 18/125,361

(22) Filed: Mar. 23, 2023

Related U.S. Application Data
(60) Provisional application No. 63/323,334, filed on Mar. 24, 2022.

(51) Int. Cl. A61M 1/16; A61M 1/00; A61M 1/28

(52) U.S. Cl. CPC A61M 1/1629; A61M 1/80; A61M 1/28; A61M 2205/3368

(57) ABSTRACT

A medical fluid system includes a medical fluid pump configured to pump a medical fluid; electronics associated with the medical fluid pump or with other components of the medical fluid system; a thermoelectric heater positioned and arranged to heat medical fluid pumped by the medical fluid pump, the thermoelectric heater including a heated side and a cooled side; a heat exchanger through which medical fluid pumped by the medical fluid pump is heated, the heat exchanger positioned and arranged so as to be in thermal communication with the heated side of the thermoelectric heater; and a mounting plate, the electronics supported by the mounting plate, the mounting plate positioned and arranged so as to be in thermal communication with the cooled side of the thermoelectric heater.

### RELATED APPLICATIONS

This application claims priority to U.S. Provisional Patent App. No. 63/323,334, entitled “DIALYSIS SYSTEM HAVING THERMOELECTRIC HEATING,” filed Mar. 24, 2022, the entire contents of which are incorporated herein by reference herein and relied upon.

### BACKGROUND

The present disclosure relates generally to medical fluid treatments and in particular to dialysis fluid treatments that require fluid heating.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Each of the above-identified dialysis modalities, except for CAPD (which typically does not involve machinery), heats the dialysis fluid prior to use for treating the patient. Online HD, online HF and online HDF each prepare the relevant dialysis and/or replacement fluids at the corresponding machine, which are heated by an inline heater prior to delivery to a dialyzer or blood line. Acute HD, HF and HDF performed at a continuous renal replacement (“CRRT”) machine typically employ bagged dialysis and replacement fluids, but which are nevertheless heated by an inline heater prior to delivery to a dialyzer or blood line.

APD systems have in many instances used batch heating of the PD fluid prior to delivery to the patient. It is known however to use inline heating for APD. One such system is set forth in U.S. Pat. No. 7,153,285, entitled, “Medical Fluid Heater Using Radiant Energy”, assigned to the assignee of the present disclosure.

Inline heating and associated heaters have a number of drawbacks Inline heaters may be expensive, require large printed circuit control boards, and require large internal space so that heat created by the heater does not overheat surrounding electronics. A larger internal volume may negatively affect the machine's design and size, usability when traveling, storage, and shipping cost. The heat generated by inline heaters may also require the dialysis machine to be provided with vents, creating openings for dust and insects to enter. The generated heat may also require the use of use high temperature grade components that drive up the cost of the dialysis machine. Moreover, the expected lifetime of the associated and surrounding electronics drops with increased surrounding temperature.

An improved way to heat any of the different dialysis treatment fluids and other medical fluids described herein is needed accordingly.

### SUMMARY

The present disclosure sets forth a medical fluid system, such as an automated peritoneal dialysis (“APD”) system, that improves dialysis fluid heating. While the present system is described primarily in connection with peritoneal dialysis, the improved dialysis fluid heating of the present disclosure applies to machines used for any dialysis modality described herein, such as online HD, HF, HDF, and acute HD, HF, HDF. The improved medical fluid heating of the present disclosure also applies to any medical fluid system in which a treatment fluid, or a patient fluid, is heated (e.g., patient blood warming).

In the APD example, the system includes an PD machine or cycler. The PD machine is capable of delivering fresh, heated PD fluid to the patient at, for example, 14 kPa (2.0 psig) or higher. The PD machine is capable of removing used PD fluid or effluent from the patient at, for example, −9 kPa (−1.3 psig) or an even greater negative pressure. The resulting PD fluid flowrate to or from the patient is dependent on a number of factors, including where the patient is located elevationally compared to the PD machine's pumping portion, the patient's sleeping position (assuming a nighttime treatment), and for a patient drain, the amount of effluent remaining in the patient's peritoneal cavity. Fresh PD fluid delivered to the patient is first heated to a body fluid temperature, e.g., 37° C.

The PD machine of the system of the present disclosure employs a thermoelectric heater, which may for example employ a Peltier module. The thermoelectric heater is considered to be a class II device, where the Peltier module, for example, is electrically isolated via ceramic plates. The thermoelectric heater may form a solid state active heat pump that transfers heat from one side of the heater element to the other side upon the consumption of electrical energy, creating a heated side and a cooled side. The heated and cooled sides are determined by the direction of electrical current flowing through the thermoelectric heater.

The heated side of the thermoelectric heater is used to heat the dialysis fluid prior to use for treatment, while the cooled side is placed in one embodiment in proximity to electronic components of the PD machine to help keep those components cool. As discussed in detail herein, the heated side is in one embodiment placed in direct thermal contact with a heat exchanger that carries the medical fluid, e.g., PD fluid, to be heated. Heat is exchanged from the heated side of the thermoelectric heater to the PD fluid flowing through the heat exchanger. In one embodiment, the cooled side of the thermoelectric heater is mounted against one or more mounting plate for the surrounding electrical components. The material of the one or more mounting plate, because it does not contact medical fluid, may be chosen for its ability to conduct thermal energy well, such as aluminum. Electrical components benefiting the most from the thermoelectric chilling, e.g., the medical or dialysis fluid pump, medical fluid valves, and associated electronics, are mounted to the one or more mounting plate. One or more heat fin may be provided to extend from the one or more mounting plate to convectively cool electrical components that cannot be mounted to the one or more mounting plate. The one or more cooled heat fin receives convective heat from the adjacent electrical components via air circulating between same. If desired, a fan may be provided to help conduct heat from the electrical components to the one or more heat fin.

Cooling the electronics also allows for lower rated and less expensive electrical components to be used. In an example, lower temperature rated electrolytic capacitors may be used when cooling the electronics, lowering component cost. Additionally, cooling the electronics prolongs the life of the electrical components. Once accepted industry rule for electrolytic capacitors (and similar for semiconductors) is that every 10° C. increase in temperature reduces component life by half.

In addition to the component cooling by the cooled side, the energy flow associated with the heated side (Qh) of the thermoelectric heater may be modeled as the energy flow on the cooled side (Qc) plus the electrical power added to the module, namely, the voltage supplied multiplied by the current supplied (V×I). The heated side energy flow (Qh), which is used to heat the PD fluid, is accordingly greater than the electrical power added to the module (V×I) by the amount of cooled side energy flow (Qc), which will be relatively large at the start of a medical fluid heating session, and which will gradually drop until the temperature difference between the heated side and the cooled side becomes too large, wherein Qc drops to zero.

One example Peltier module useable with the thermoelectric heater of the present disclosure is able to handle a heated side versus cooled side temperature difference of 75° C., which means that Qc is greater than zero as long as the source from which energy is taken (the cooled side) is larger than the medical or dialysis fluid temperature exiting the heat exchanger. The PD fluid temperature exiting the heat exchanger is in one embodiment body fluid temperature, e.g., 37° C., such that additional heat (Qc) from the cold side is provided as long as the cold side remains warmer than −38° C., which is highly likely especially considering that the cold side is exchanging heat with surrounding electrical components, cooling such components.

The solid state nature of the thermoelectric heater of the present disclosure provides a quiet solution because there is no internal switching. The powering of the thermoelectric heater is also advantageous because the heater uses direct current (“DC”) power, which may be supplied by a backup battery instead of or in addition to DC power from the main power supply. The battery power may be provided additionally and intermittently, e.g., in times of maximum power draw and high heating power modes, so as not to overstress the main power supply. The clean DC power also reduces electrical magnetic interference (“EMI”) and radio frequency (“RF”) noise as compared to 115/230 VAC heater elements. Even if a pulse width modulation (“PWM”) driver is used to drive the thermoelectric heater, which is contemplated, EMF and RF noise are lower due to switching at a significantly lower voltage. The use of DC power also reduces leakage current and flicker since power is gradually controlled and large resistive loads are not switched on and off as is the case with many resistive inline heaters.

Also, since the polarity of the power delivered to the thermoelectric heater can be reversed, it is contemplated to reverse the polarity when the cooled side temperature is approaching a temperature that is deemed to be too low, such as a dew point temperature, here with the goal of preventing condensation from forming on the cold side of the module. To this end, an inexpensive humidity/temperature sensor integrated circuit may be provided on a printed circuit board of the control unit of the present system, wherein the sensor and integrated circuit are configured to calculate the dew point temperature. Another reason to switch polarity and thus the heated and cooled sides of the thermoelectric heater is in the case of a no-medical-fluid-flow situation where polarity is reversed to avoid medical fluid overheating and to reduce the amount of discarded medical fluid.

It is contemplated to use the thermoelectric heater of the present disclosure as a replacement for a typical resistive inline heater. It is also contemplated to use the thermoelectric heater in combination with a smaller typical resistive inline heater, which mitigates against the drawbacks of same, e.g., heater expense, heat generation, spacing requirements, and expensive electrical components. In an embodiment, the thermoelectric heater is placed upstream of the smaller resistive inline heater so that the thermoelectric heater is put to full use. The smaller resistive inline heater may then almost be used as a backup heater only when needed to supply any heating delta needed for the medical or dialysis fluid to reach the desired treatment temperature. It is expressly contemplated however to reverse the order of the thermoelectric heater and the smaller resistive inline heater, so that the resistive heater is placed upstream of the thermoelectric heater, wherein the polarity of thermoelectric heater may be reversed, e.g., to cool overheated medical fluid exiting the resistive heater.

In light of the disclosure set forth herein, and without limiting the disclosure in any way, in a first aspect of the present disclosure, which may be combined with any other aspect, or portion thereof, a medical fluid system include a medical fluid pump configured to pump a medical fluid; a thermoelectric heater positioned and arranged to heat medical fluid pumped by the medical fluid pump, the thermoelectric heater including a heated side and a cooled side; a heat exchanger through which medical fluid pumped by the medical fluid pump is heated, the heat exchanger positioned and arranged so as to be in thermal communication with the heated side of the thermoelectric heater; and a mounting plate, the medical fluid pump or other components of the medical fluid system supported by the mounting plate, the mounting plate positioned and arranged so as to be in thermal communication with the cooled side of the thermoelectric heater.

In a second aspect of the present disclosure, which may be combined with any other aspect, or portion thereof, the heat exchanger being in thermal communication with the heated side of the thermoelectric heater includes directly contacting the heated side.

In a third aspect of the present disclosure, which may be combined with any other aspect, or portion thereof, the heat exchanger includes a conductive heat exchanger block and a conductive serpentine pathway supported by the conductive heat exchanger block.

In a fourth aspect of the present disclosure, which may be combined with any other aspect, or portion thereof, the conductive heat exchanger block is made of aluminum or copper and the conductive serpentine pathway is made of stainless steel.

In a fifth aspect of the present disclosure, which may be combined with any other aspect, or portion thereof, the medical fluid system includes a temperature sensor located on or inside the conductive heat exchanger block.

In a sixth aspect of the present disclosure, which may be combined with any other aspect, or portion thereof, the mounting plate being in thermal communication with the cooled side of the thermoelectric heater includes being in direct contact with the cooled side.

In a seventh aspect of the present disclosure, which may be combined with any other aspect, or portion thereof, the medical fluid pump or other components of the medical fluid system being supported by the mounting plate includes the medical fluid pump or other components of the medical fluid system being mounted to the mounting plate.

In an eighth aspect of the present disclosure, which may be combined with any other aspect, or portion thereof, the other components include at least one of (i) at least one valve, (ii) at least one temperature sensor, (iii) at least one pressure sensor, or (iv) a flow sensor or flow switch, and electronics associated with the other components.

In a ninth aspect of the present disclosure, which may be combined with any other aspect, or portion thereof, the medical fluid system includes at least one heat pipe extending from the medical fluid pump or at least one of the other components of the medical fluid system to the mounting plate for conducting heat to the mounting plate.

In a tenth aspect of the present disclosure, which may be combined with any other aspect, or portion thereof, the mounting plate is attached to or is formed to have at least one heat fin for convectively transferring heat from at least one component of the medical fluid system to the mounting plate, the at least one component located adjacent to the at least one heat fin.

In an eleventh aspect of the present disclosure, which may be combined with any other aspect, or portion thereof, the medical fluid system includes at least one fan positioned and arranged to blow air between the at least one heat fin and the at least one convectively cooled component of the medical fluid system.

In a twelfth aspect of the present disclosure, which may be combined with any other aspect, or portion thereof, the thermoelectric heater includes a plurality of semiconductors extending between the heated side and a cooled side.

In a thirteenth aspect of the present disclosure, which may be combined with any other aspect, or portion thereof, the medical fluid system includes a plurality of conductive leads located between the plurality of semiconductors and the heated and cooled sides.

In a fourteenth aspect of the present disclosure, which may be combined with any other aspect, or portion thereof, the plurality of conductive leads are positioned and arranged such that the plurality of semiconductors operate electrically in series.

In a fifteenth aspect of the present disclosure, which may be combined with any other aspect, or portion thereof, the plurality of semiconductors operate thermally in parallel.

In a sixteenth aspect of the present disclosure, which may be combined with any other aspect, or portion thereof, the medical fluid system includes a resistive inline heater located fluidically in series with the thermoelectric heater.

In a seventeenth aspect of the present disclosure, which may be combined with any other aspect, or portion thereof, the medical fluid system includes a control unit, the thermoelectric heater and the resistive inline heater under control of the control unit, the control unit configured to power the resistive inline heater as needed to aid the thermoelectric heater in heating the medical fluid to a desired temperature.

In an eighteenth aspect of the present disclosure, which may be combined with any other aspect, or portion thereof, the medical fluid system includes a control unit, the control unit configured to cause the polarity of power to the thermoelectric heater to be reversed such that the heated side becomes a cooled side of the thermoelectric heater, wherein the heat exchanger is then positioned and arranged so as to be in thermal communication with the cooled side.

In a nineteenth aspect of the present disclosure, which may be combined with any other aspect, or portion thereof, the medical fluid system includes a humidity sensor positioned and arranged to measure humidity adjacent to the mounting plate, the control unit further configured to use an output from the humidity sensor to determine when to cause the polarity of power to the thermoelectric heater to be reversed so as to prevent the mounting plate from reaching a dew point temperature.

In a twentieth aspect of the present disclosure, which may be combined with any other aspect, or portion thereof, a method for heating a medical fluid includes supplying electrical energy to a thermoelectric heater so as to create a heated side and a cooled side of the thermoelectric heater, the thermoelectric heater provided as part of a medical fluid machine; locating a heat exchanger so that medical fluid flowing within the heat exchanger receives heat from the heated side of the thermoelectric heater; and locating at least one component of the medical fluid machine so that heat given off by the at least one component is received by the cooled side of the thermoelectric heater, the heat received by the cooled side of the thermoelectric heater adding to available heat at the heated side of the thermoelectric heater generated via the supply of electrical energy.

In a twenty-first aspect of the present disclosure, which may be combined with any other aspect, or portion thereof, the medical fluid carrying heat exchanger is reusable or is provided as part of a disposable medical fluid carrying set.

In a twenty-second aspect of the present disclosure, which may be combined with any other aspect, or portion thereof, locating the heat exchanger includes locating the heat exchanger within the medical fluid machine or as part of an actuating surface of the medical fluid machine.

In a twenty-third aspect of the present disclosure, which may be combined with any other aspect, or portion thereof, the medical fluid heating method includes reversing the polarity of electrical energy to the thermoelectric heater, reversing the heated side and the cooled side of the thermoelectric heater, so that medical fluid flowing within the heat exchanger delivers heat to the cooled side of the thermoelectric heater.

In a twenty-fourth aspect of the present disclosure, which may be combined with any other aspect, or portion thereof, any of the features, functionality and alternatives described in connection with any one or more of FIGS. 1 to 3 may be combined with any of the features, functionality and alternatives described in connection with any other of FIGS. to 3.

In light of the above aspects and present disclosure set forth herein, it is an advantage of the present disclosure to provide a medical fluid heating system that involves thermoelectric cooling.

It is another advantage of the present disclosure to provide a medical fluid heating system that reduces electrical component damage due to overheating via heat from the medical fluid heater, reducing thermal stress via a more even temperature profile for the electrical components over the course of treatment and post-treatment disinfection, and helping with mean time between failure (“MTBF”) of the electrical components.

It is a further advantage of the present disclosure to provide a medical fluid heating system that is able to cool surrounding electrical components, which improves component reliability and allows less expensive electrical components to be used in certain instances.

Moreover, it is an advantage of the present disclosure to provide a medical fluid heating system that is able to use heat from surrounding electronics to increase the heating potential of the medical fluid heater.

It is yet another advantage of the present disclosure to provide a medical fluid heating system that operates quietly.

It is yet a further advantage of the present disclosure to provide a medical fluid heating system that is able to switch heated and cooled sides of the thermoelectric heating module in the case of a no-medical-fluid-flow situation to avoid medical fluid overheating and to reduce the amount of discarded medical fluid.

It is yet a further advantage of the present disclosure to provide a medical fluid heating system that reduces at least one of electrical magnetic interference (“EMI”), radio frequency (“RF”) noise, leakage current, and flicker relative to those produced by VAC voltage resistive heater elements.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system having the thermoelectric heating of the present disclosure.

FIG. 2 is an elevation view showing the inside of a medical fluid, e.g., PD fluid, machine having an embodiment for employing the thermoelectric heating of the present disclosure.

FIG. 3 is a perspective view of one embodiment for a medical fluid carrying heat exchanger useable with the thermoelectric heating of the present disclosure.

### DETAILED DESCRIPTION

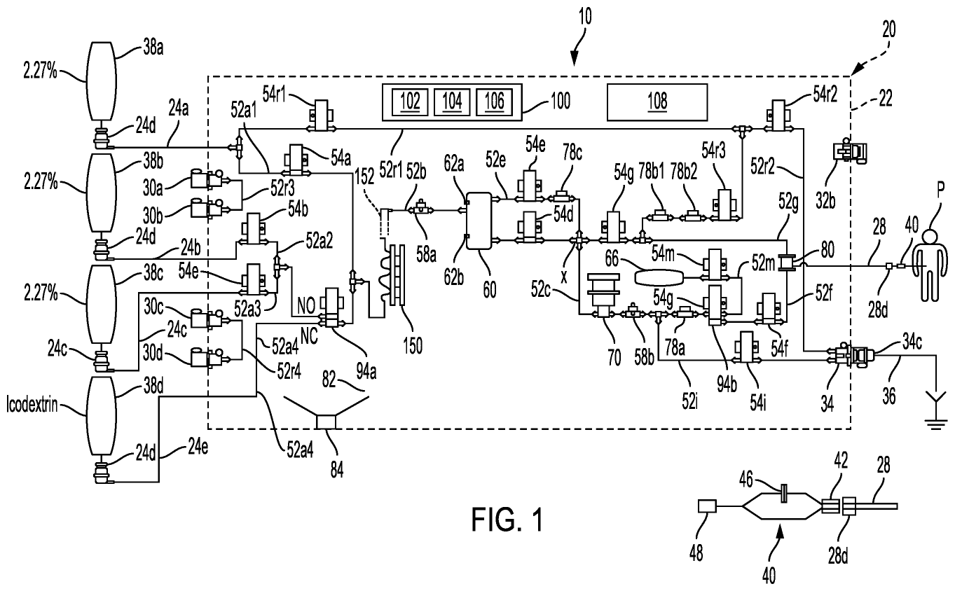

Referring now to the drawings and in particular to FIG. 1, an example medical fluid system that may employ the thermoelectric heating of the present disclosure is illustrated by peritoneal dialysis (“PD”) system 10. System 10 includes a PD machine or cycler 20 and a control unit 100 having one or more processor 102, one or more memory 104, video controller 106 and user interface 108. Control unit 100 controls all electrical fluid flow and heating components of system 10 and receives outputs from all sensors of system 10. System 10 in the illustrated embodiment includes durable and reusable components that contact medical fluid, such as PD fluid, which necessitates that PD machine or cycler 20 be disinfected between treatments, e.g., via heat disinfection.

System 10 in FIG. 1 includes an inline thermoelectric heater 150 (discussed in detail below), reusable supply lines or tubes 52a1 to 52a4 and 52b, air trap 60 operating with respective upper and lower level sensors 62a and 62b, air trap valve 54d, vent valve 54e located along vent line 52e, reusable line or tubing 52c, dialysis fluid pump 70, temperature sensors 58a and 58b, reusable line or tubing 52d, pressure sensors 78a, 78b1, 78b2 and 78c, reusable patient tubing or lines 52f and 52g having respective valves 54f and 54g, dual lumen reusable patient line 28, hose reel 80 for retracting patient line 28, reusable drain tubing or line 52i extending to drain line connector 34 and having a drain line valve 54i, and reusable recirculation disinfection tubing or lines 52r1 and 52r2 operating with respective disinfection valves 54r1 and 54r2. A third recirculation or disinfection tubing or line 52r3 extends between disinfection connectors 30a and 30b for use during disinfection. A fourth recirculation or disinfection tubing or line 52r4 extends between disinfection connectors 30c and 30d for use during disinfection.

System 10 further includes PD fluid containers or bags 38a to 38c (e.g., holding the same or different formulations of PD fluid), which connect to distal ends 24d of reusable PD fluid lines 24a to 24c, respectively. System 10d further includes a fourth PD fluid container or bag 38d that connects to a distal end 24d of reusable PD fluid line 24e. Fourth PD fluid container or bag 38d may hold the same or different type (e.g., icodextrin) of PD fluid than provided in PD fluid containers or bags 38a to 38c. Reusable PD fluid lines 24a to 24c and 24e extend in one embodiment through apertures (not illustrated) defined or provided by housing 22 of cycler 20.

System 10 in the illustrated embodiment includes four disinfection connectors 30a to 30d for connecting to distal ends 24d of reusable PD fluid lines 24a to 24c and 24e, respectively, during disinfection. System 10 also provides patient line connector 32 that includes an internal lumen, e.g., a U-shaped lumen, which directs fresh or used dialysis fluid from one PD fluid lumen of dual lumen reusable patient line 28 into the other PD fluid lumen. Reusable supply tubing or lines 52a1 to 52a4 communicate with reusable supply lines 24a to 24c and 24e, respectively. Reusable supply tubing or lines 52a1 to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

FIG. 1 also illustrates that system 10 includes and uses disposable filter set 40, which communicates fluidly with the fresh and used PD fluid lumens of dual lumen reusable patient line 28. Disposable filter set 40 includes a disposable connector 42 that connects to distal end 28d of reusable patient line 28. Disposable filter set 40 includes a connector 48 that connects to the patient's transfer set. Disposable filter set 40 further includes a sterilizing grade filter membrane 46 that further filters fresh PD fluid.

System 10 is constructed in one embodiment such that drain line 52i during filling is fluidly connected downstream from dialysis fluid pump 70. In this manner, if drain valve 54i fails or somehow leaks during a patient fill of patient P, fresh PD fluid is pushed down disposable drain line 36 instead of used PD fluid potentially being pulled into pump 70. Disposable drain line 36 is in one embodiment removed for disinfection, while drain line connector 34 is capped via a cap 34c.

System 10 further includes a leak detection pan 82 located at the bottom of housing 22 of cycler 20 and a corresponding leak detection sensor 84 outputting to control unit 100. In the illustrated example, system 10 is provided with an additional pressure sensor 78c located upstream of dialysis fluid pump 70, which allows for the measurement of the suction pressure of pump 70 to help control unit 100 to more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control, as discussed herein, while the output of the other pressure sensor is a safety or watchdog output to make sure the control pressure sensor is reading accurately. Pressure sensors 78b1 and 78b2 are located along a line including a third recirculation valve 54r3. In still a further example, system 10 may employ one or more cross, marked via an X in FIG. 1, which may (i) reduce the overall amount and volume of the internal, reusable tubing, (ii) reduce the number of valves needed, and (iii) allow the portion of the fluid circuitry shared by both fresh and used PD fluid to be minimized.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is instead used during disinfection.

FIG. 1 illustrates that in an optional embodiment, thermoelectric heater 150, under control of control unit 100, is used in combination with a smaller typical resistive inline heater 152 (shown in phantom line as being optional) also under control of control unit 100, which mitigates against the drawbacks of such inline heaters, e.g., heater expense, heat generation, venting space, and expensive electrical components, etc. In the illustrated embodiment, thermoelectric heater 150 is placed upstream of smaller resistive inline heater 152 so that the thermoelectric heater is put to full use. Smaller resistive inline heater 152 may then be used as a backup heater only when needed to supply any heating delta so that the medical or dialysis fluid reaches the desired treatment temperature. It is expressly contemplated however to reverse the order of thermoelectric heater 150 and smaller resistive inline heater 152 as shown in FIG. 1, so that resistive heater 152 is placed upstream of thermoelectric heater 150 (not illustrated). In the non-illustrated order where resistive heater 152 is placed upstream of thermoelectric heater 150, it is contemplated for control unit 100 to reverse the polarity of thermoelectric heater 150 if needed to cool overheated medical fluid exiting resistive heater 152. It is contemplated in one embodiment here to run smaller resistive inline heater 152 at a lower duty cycle under normal circumstances to pre-warm the medical fluid, which reduces the burden on downstream thermoelectric heater 150 in heating the medical fluid to a desired treatment temperature.

Although not illustrated, it is contemplated to place a temperature sensor outputting to control unit 100 between thermoelectric heater 150 and inline heater 152 (in either order, thermoelectric heater 150 first or inline heater 152 first). The additional temperature sensor senses the amount of heat delivered by thermoelectric heater 150. Control unit 100 may then use the output from the additional temperature sensor to determine when and how much (e.g., duty cycle) to power inline heater 152. In an alternative situation in which inline heater 152 is used continuously or almost continuously in combination with the continuous use of thermoelectric heater 150, the additional temperature sensor may not be needed and the output from temperature sensor 58a may be used as feedback, e.g., in a proportional, integral, derivative (“PID”) routine, to control both thermoelectric heater 150 and inline heater 152.

It should be appreciated that system 10 is not required to (i) be a dialysis system, or (ii) use redundant or durable components that are disinfected between uses to employ the sensor thermoelectric heating of the present disclosure. System 10 may instead be any type of medical fluid system and may employ a disposable set having a disposable pumping portion that contacts the corresponding medical fluid. In the primary example described herein, thermoelectric heater 150 is described as operating with PD machine or cycler 20 for which the PD fluid heat exchanger is a reusable or durable component that is disinfected between treatments.

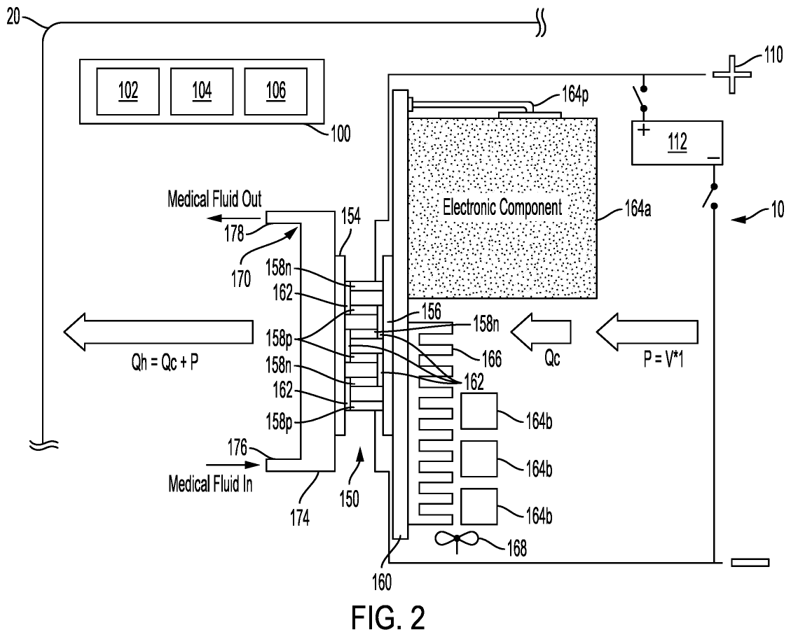

Referring now to FIG. 2, an embodiment for providing thermoelectric heater 150 within PD machine or cycler 20 of system 10 is illustrated. Thermoelectric heater 150 in one embodiment operates according to a Peltier effect and thus may be referred to herein as a Peltier module 150. Thermoelectric heater 150 in FIG. 2 includes two sides or plates 154 and 156, which are separated by n-type semiconductors 158n and p-type semiconductors 158p in one embodiment. When a direct current (“DC”) voltage, such as a 5 VDC, 12 VDC, or 24 VDC depending on the type of thermoelectric heater 150, supplied by power supply 110 is applied to and flows through sides 154, 156 and semiconductors 158n, 158p, heat is conducted from cooled side or plate 156 to heated side or plate 154. Power supply 110 is under the control of control unit 100 in one embodiment. The polarity of the DC voltage applied to sides 154, 156 and semiconductors 158n, 158p may be reversed by control unit 100, such that heat is conducted instead from side or plate 154 to side or plate 156.

The input voltage level for thermoelectric heater 150 (e.g., 5 VDC, 12 VDC, or 24 VDC) may be based on its maximum current rating, which should not be exceeded. Since the resistance thermoelectric heater 150 is relatively fixed (varies some with temperature), the voltage applied is adjusted in one embodiment to provide a current lower than the maximum current rating. The adjustment of voltage is performed via feedback control by control unit 100 using the output from temperature sensor 58a in one embodiment, wherein the output of the feedback control sets the input voltage at a level that maintains the resulting current below the maximum threshold.

Heated side or plate 154 and cooled side or plate 156 are ceramic in one embodiment, but may be made of other conductive materials and combinations thereof. Unique n-type and one p-type semiconductors 158n, 158p may be used because they provide different electron densities. Alternating n-type and p-type pillars of semiconductors 158n, 158p are placed thermally in parallel to each other and electrically in series with each other in one embodiment. The n-type and p-type semiconductors 158n, 158p may be connected electrically in series with heated side 154 and cooled side 156 via conductive leads 162, e.g., aluminum or copper leads, located between the ends of the semiconductor pillars and the insides of heated side 154 and cooled side 156.

When a power supply 110 of machine 20 applies the DC voltage to the free ends of n-type and p-type semiconductors 158n, 158p, DC current flows across the semiconductors and conductive leads 162 connecting the semiconductors in series, causing the temperature difference between heated side 154 and cooled side 156. Cooled side 156 absorbs heat which is then transported by semiconductors 158n, 158p, to heated side 154. The heating ability of thermoelectric heater 150 is in one embodiment proportional to the combined total cross-sectional area of the pillars of each semiconductor 158n, 158p (e.g., connected in series electrically to reduce the supply current). The length of the pillars semiconductors 158n, 158p is in one embodiment selected based on a balance between (i) longer pillars, which have a greater thermal resistance between the sides 154, 156 but produce more resistive heating, and (ii) shorter pillars, which have a greater electrical efficiency but let more heat leak from the hot to the cold side by thermal conduction. While system 10 illustrates a single thermoelectric heater 150, multiple thermoelectric heaters or Peltier modules 150, each operating with medical fluid heat exchanger 170, may be provided instead. Here, power supply 110 powers each thermoelectric heater or Peltier module 150 separately.

Thermoelectric heater 150 may be considered to be a class II device, where the Peltier module, for example, is electrically isolated via ceramic sides or plates 154, 156, limiting electrical creepage. Thermoelectric heater 150 forms a solid state active heat pump that transfers heat from cooled side 156 to heated side 154. It should be appreciated that control unit 100 and power supply 110 are configured in one embodiment to be able to reverse the polarity of the DC power applied, switching the thermal polarities such that side 154 becomes the cooled side and side 156 becomes the heated side. The heated and cooled sides are thus determined by the direction of electrical current flowing through thermoelectric heater 150.

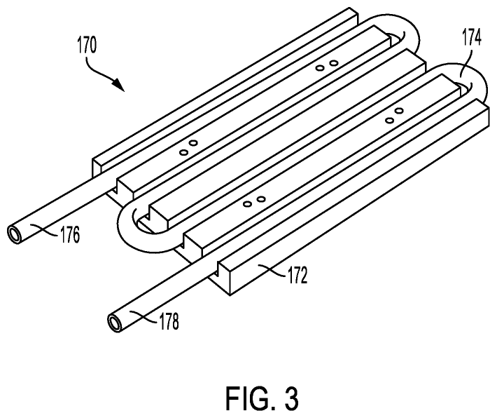

System 10 in FIG. 2 illustrates that heated side 154 of thermoelectric heater 150 is used to heat medical fluid, such as dialysis fluid, prior to use for treatment, while cooled side 156 is placed in proximity to electronic components of medical fluid machine 20 to help keep those components cool. As illustrated in FIG. 2, heated side 154 is in one embodiment placed in direct thermal contact with a heat exchanger 170 that carries medical fluid, e.g., PD fluid, to be heated. Heat is exchanged from heated side 154 of thermoelectric heater 150 to PD fluid flowing through heat exchanger 170. FIG. 2 illustrates heat exchanger 170 schematically, while FIG. 3 illustrates one implementation for heat exchanger 170. FIGS. 2 and 3 illustrate that heat exchanger 170 in one embodiment includes a heat exchange block 172, which may be a conductive metal block, such as aluminum, copper or stainless steel. Heat exchange block 172 does not contact medical fluid, so its material may be optimized for thermal conductivity.

FIG. 3 illustrates that heat exchanger 170 includes a conductive serpentine pathway 174 that carries medical fluid, such as dialysis fluid. Conductive serpentine pathway 174 is accordingly made of medical fluid safe stainless steel in one embodiment. Serpentine pathway 174 enables the medical fluid to travel back and forth along heated side 154 and heat exchange block 172 multiple times. In system 10 of FIG. 2, heat conducts accordingly from heated side 154 to heat exchange block 172, from heat exchange block 172 to serpentine pathway 174, and from serpentine pathway 174 to the medical fluid. Serpentine pathway 174 includes a medical fluid inlet 176 and a medical fluid outlet 178, which extend to other fluid components as illustrated in FIG. 1. In an alternative embodiment, the serpentine pathway is formed directly in heat exchange block 172, which may include two sealed and machined stainless steel halves forming the serpentine pathway or may be formed via an additive process so as to have the serpentine pathway. In a further alternative embodiment, heat exchange block 172 is not provided and conductive serpentine pathway 174 is instead abutted directly against heated side 154 of thermoelectric heater 150.

It is contemplated to mount temperature sensor 58a (FIG. 1) or an additional temperature sensor on or inside block 172, the output of which may be used for medical fluid, e.g., PD fluid, temperature control and to allow block 172 to be preheated to a desired temperature prior to treatment. Such preheating decreases startup time and minimizes the possibility of having initially cold fluid. The output of temperature sensor 58a (FIG. 1) or an additional temperature sensor located on or inside block 172 (FIG. 3) may be used additionally to allow block 172 to be cooled to a desired temperature. Being able to cool block 172 and thus the overheated medical fluid allows for the overheated medical fluid to be cooled and then delivered for treatment, whereas in many resistive inline heater applications, overheated medical fluid is delivered instead to drain, wasting such fluid.

In the illustrated embodiment of FIG. 2, the cooled side 156 of thermoelectric heater 150 is mounted or otherwise abutted against one or more mounting plate 160 for mounting electrical components 164a, which may be any of the components illustrated in FIG. 1. The material of one or more mounting plate 160, because it does not contact medical fluid, may be chosen for its ability to conduct thermal energy well, such as aluminum. Any electrical components 164a benefiting from the cooling of thermoelectric heater 150, e.g., medical or dialysis fluid pump 70, medical fluid valves 54a to 54g, 54i, 54m, 54r1 to 54r3, 94a, 94b, flow sensor or flow switch 26, temperature sensors 58a, 58b, pressure sensors 78a, 78b1, 78b2, 78c, any associated electronics, and any other electronics of medical fluid machine 20, may be mounted to one or more mounting plate 160. Any electrical component 164a whose mounting surface is not thermally conductive, e.g., if insulation is provided, may be thermally aided via one or more heat pipe 164p that extends from a conductive portion of the component 164a to one or more mounting plate 160. Heat pipe 164p may be made of any thermally conductive material, such as aluminum or copper.

FIG. 2 further illustrates that one or more heat fin 166 may be attached to or formed with one or more mounting plate 160 to convectively cool electrical components 164b (e.g., any of the components listed above, associated electronics, or other electronics) that for whatever reason cannot be mounted to the one or more mounting plate. One or more cooled heat fin 166 receives heat from adjacent electrical components 164b via air circulating between same. If desired, a fan 168 under control of control unit 100 may be provided to help convect heat from electrical components 164b to one or more heat fin 166. Fan 168 or a different fan under control of control unit 100 may also be oriented and be run at a low speed to cool the environment at the dry side of medical fluid machine 20, e.g., to the right of mounting plate 160 in FIG. 2. Fan 168 or a different fan under control of control unit 100 may also be oriented to cool a desired surface of medical fluid machine 20. Additionally, any fan, such as fan 168, may be mounted within an aperture provided at the surface of medical fluid machine 20 to draw in ambient air. Further additionally, any fan, such as fan 168, may be mounted to an outer surface of medical fluid machine 20, assuming Class II certification of medical fluid machine 20 can be met, which may be accomplished by electrically isolating the surface of medical fluid machine 20 that is used for mounting from all medical fluid flowpaths and electronics (examples listed herein) of medical fluid machine 20.

The energy flow (Qh) associated with heated side 154 of thermoelectric heater 150 may be modeled as the energy flow (Qc) on the cooled side 156 plus the electrical power added to module 150, namely, the voltage supplied multiplied by the current supplied (V×I). Heated side 154 energy flow (Qh), which is used to heat the medical or PD fluid, is accordingly greater than the electrical power added to the module (V×I) by the amount of cooled side 156 energy flow (Qc), which will be relatively large at the start of a medical fluid heating session, and which will gradually drop until the temperature difference between heated side 154 and cooled side 156 becomes too large, wherein Qc drops to zero.

One example Peltier module useable with thermoelectric heater 150 of the present disclosure is able to handle a heated side 154 versus cooled side 156 temperature difference of 75° C. For a PD fluid treatment where PD fluid exits the heat exchanger at, e.g., 37° C., Qc will contribute energy (as long as the temperature difference between heated side 154 and cooled side 156 is not too large) from the cold side 156 as long as the cold side remains warmer than −38° C. This is highly likely especially considering that cold side 156 is exchanging heat with surrounding electrical components 164a, 164b, cooling such components.

A sample calculation of the energy taken from the surroundings, including electrical components 164a, 164b operating under standard conditions may be as follows, wherein the following assumptions are made regarding mass and specific heat for certain components:
mounting plate(s) 160, aluminum, 0.5 kg, 900 J/(kg*° C.),
printed circuit board (“PCB”), 0.5 kg, 390 J/(kg*° C.),
valve, 0.25 kg each, 400 J/(kg*° C.),
water, 2 kg, 4180 J/(kg*° C.) (approximating medical fluid)
   If it is assumed that 2 kg of water (medical fluid) is heated from 15° C. to 37° C. during a patient fill, 4180 J/(kg*° C.)*2 kg*(37-15° C.)=183920 J of energy is required. If it is assumed that medical fluid machine 20 holds medical fluid at 23° C. (fluid now at room temperature) at the start of the patient fill and that energy from the mounting plate(s) 160 and components 164a, 164b is allowed to be used down to 8° C., the following amount of energy is provided: (900 J/(kg*° C.)*0.5 kg+390 J/(kg*° C.)*0.5 kg+400 J/(kg*° C.)*0.25 kg*10 valves for example)*15° C.=24675 J. The 24675 J is in one example the energy taken from a PD machine or cycler 20 and provided to thermoelectric heater 150 during a patient fill. A fully loaded medical fluid machine 20 has more mass than the small amount listed above (e.g., roughly 10 kg), so that the actual amount of energy that may be borrowed from medical fluid machine 20 would be higher. The pure thermal mass of the machine 20 should provide at least 24675/183920 or 13% of the energy needed. Active components 164a, 164b (valves and dialysis fluid pump 70) also generate heat while running, thus increasing the available energy.

The solid state nature of thermoelectric heater 150 of the present disclosure provides a quiet solution because there is no internal switching. The powering of thermoelectric heater 150 is also advantageous because the heater uses DC power, which may be supplied by a backup battery 112 (FIG. 2) instead of or in addition to DC power from main power supply 110. The power from backup battery 112 may be provided additionally and intermittently, e.g., in times of maximum power draw and high heating power modes, so as not to overstress main power supply 110. Also, since the polarity of the power delivered to thermoelectric 150 heater can be reversed, it is contemplated for control unit 100 to reverse the polarity of power from supply 110 when the temperature of cooled side 156 approaches a dew point temperature with the goal of preventing condensation from forming on the cold side of module 150. To this end, an inexpensive humidity/temperature sensor integrated circuit (not illustrated) may be provided on a printed circuit board of control unit 100, wherein the sensor and integrated circuit are configured to calculate the dew point temperature.

It is contemplated to user a pulse width modulation (“PWM”) driver to control input power to thermoelectric heater 150. Even so, electrical magnetic interference (“EMI”) and radio frequency (“RF”) noise are lower than for resistive heaters running on 115/230 VAC due to switching at a significantly lower DC voltage. The use of DC power also reduces leakage current and flicker since power is gradually controlled and large resistive loads are not switched on and off as is the case with many resistive inline heaters.

It should be understood that various changes and modifications to the presently preferred embodiments described herein will be apparent to those skilled in the art. It is therefore intended that such changes and modifications be covered by the appended claims. For example, as mentioned above, system 10 does not have to use redundant or durable components and may instead employ a disposable set having a disposable pumping portion that contacts the corresponding medical fluid. Here, serpentine pathway 174 may be a disposable pathway formed as part of the disposable set, which is held removably by heat exchange block 172 during treatment and removed from heat exchange block 172 after treatment. Here, heat exchange block 172 may be provided as part of a front (or top) actuation surface of medical fluid machine 20 and disposable serpentine pathway 174 may be held in place within heat exchange block 172 via a hinged door of the medical fluid machine 20. In an alternative disposable embodiment, heat exchange block 172 and serpentine pathway 174 are formed together as part of the disposable set. Here, heated side 154 of thermoelectric heater 150 may be provided as part of a front (or top) actuation surface of medical fluid machine 20 and disposable heat exchange block 172 and serpentine pathway 174 may be held in place against heated side 154 via a hinged door of the medical fluid machine 20.

The invention is claimed as follows:

1. A medical fluid system comprising:
a medical fluid pump configured to pump a medical fluid;
a thermoelectric heater positioned and arranged to heat medical fluid pumped by the medical fluid pump, the thermoelectric heater including a heated side and a cooled side;
a heat exchanger through which medical fluid pumped by the medical fluid pump is heated, the heat exchanger positioned and arranged so as to be in thermal communication with the heated side of the thermoelectric heater; and
a mounting plate, the medical fluid pump or other components of the medical fluid system supported by the mounting plate, the mounting plate positioned and arranged so as to be in thermal communication with the cooled side of the thermoelectric heater.

2. The medical fluid system of claim 1, wherein the heat exchanger being in thermal communication with the heated side of the thermoelectric heater includes directly contacting the heated side.

3. The medical fluid system of claim 1, wherein the heat exchanger includes a conductive heat exchanger block and a conductive serpentine pathway supported by the conductive heat exchanger block.

4. The medical fluid system of claim 3, wherein the conductive heat exchanger block is made of aluminum or copper and the conductive serpentine pathway is made of stainless steel.

5. The medical fluid system of claim 3, which includes a temperature sensor located on or inside the conductive heat exchanger block.

6. The medical fluid system of claim 1, wherein the mounting plate being in thermal communication with the cooled side of the thermoelectric heater includes being in direct contact with the cooled side.

7. The medical fluid system of claim 1, wherein the medical fluid pump or other components of the medical fluid system being supported by the mounting plate includes the medical fluid pump or other components of the medical fluid system being mounted to the mounting plate.

8. The medical fluid system of claim 1, wherein the other components include at least one of (i) at least one valve, (ii) at least one temperature sensor, (iii) at least one pressure sensor, or (iv) a flow sensor or flow switch, and electronics associated with the other components.

9. The medical fluid system of claim 1, which includes at least one heat pipe extending from the medical fluid pump or at least one of the other components of the medical fluid system to the mounting plate for conducting heat to the mounting plate.

10. The medical fluid system of claim 1, wherein the mounting plate is attached to or is formed to have at least one heat fin for convectively transferring heat from at least one component of the medical fluid system to the mounting plate, the at least one component located adjacent to the at least one heat fin.

11. The medical fluid system of claim 10, which includes at least one fan positioned and arranged to blow air between the at least one heat fin and the at least one convectively cooled component of the medical fluid system.

12. The medical fluid system of claim 1, wherein the thermoelectric heater includes a plurality of semiconductors extending between the heated side and a cooled side.

13. The medical fluid system of claim 12, which includes a plurality of conductive leads located between the plurality of semiconductors and the heated and cooled sides.

14. The medical fluid system of claim 13, wherein the plurality of conductive leads are positioned and arranged such that the plurality of semiconductors operate electrically in series.

15. The medical fluid system of claim 12, wherein the plurality of semiconductors operate thermally in parallel.

16. The medical fluid system of claim 1, which includes a resistive inline heater located fluidically in series with the thermoelectric heater.

17. The medical fluid system of claim 16, which includes a control unit, the thermoelectric heater and the resistive inline heater under control of the control unit, the control unit configured to power the resistive inline heater as needed to aid the thermoelectric heater in heating the medical fluid to a desired temperature.

18. The medical fluid system of claim 1, which includes a control unit, the control unit configured to cause the polarity of power to the thermoelectric heater to be reversed such that the heated side becomes a cooled side of the thermoelectric heater, wherein the heat exchanger is then positioned and arranged so as to be in thermal communication with the cooled side.

19. The medical fluid system of claim 18, which includes a humidity sensor positioned and arranged to measure humidity adjacent to the mounting plate, the control unit further configured to use an output from the humidity sensor to determine when to cause the polarity of power to the thermoelectric heater to be reversed so as to prevent the mounting plate from reaching a dew point temperature.

20. A method for heating a medical fluid comprising:
supplying electrical energy to a thermoelectric heater so as to create a heated side and a cooled side of the thermoelectric heater, the thermoelectric heater provided as part of a medical fluid machine;
locating a heat exchanger so that medical fluid flowing within the heat exchanger receives heat from the heated side of the thermoelectric heater; and
locating at least one component of the medical fluid machine so that heat given off by the at least one component is received by the cooled side of the thermoelectric heater, the heat received by the cooled side of the thermoelectric heater adding to available heat at the heated side of the thermoelectric heater generated via the supply of electrical energy.
